(19) United States
(12) Patent Application Publication
(10) Pub. No.: US 2020/0188351 A1
(43) Pub. Date: Jun. 18, 2020

(54) TOPICAL HAIR AND SKIN FORMULATION

(71) Applicant: Andreau Rico Francisco Coto, Cheyenne, WY (US)

(72) Inventor: Andreau Rico Francisco Coto, Cheyenne, WY (US)

(21) Appl. No.: 16/510,693

(22) Filed: Jul. 12, 2019

Related U.S. Application Data
(60) Provisional application No. 62/697,239, filed on Jul. 12, 2018.

(51) Int. Cl. A61K 31/355; A61K 36/36; A61K 36/889; A61K 36/54; A61K 36/185; A61K 31/25; A61K 9/00

(52) U.S. Cl. CPC A61K 31/355; A61K 36/36; A61K 36/889; A61K 9/0014; A61K 36/185; A61K 31/25; A61K 36/54

(57) ABSTRACT

A topical hair and skin formulation which has primarily medicinal and healing properties, but may also be used to moisturize skin and dress or style hair of all types. The formulation is a blend of oils each having various individual properties. The invented oil blend keeps connective tissues strong and supple while protecting against the breakdown of the skin's cell structure and the hair's follicle and inner cuticle with essential vitamins, minerals, amino and fatty acids implementing healing and regeneration. The invented oil blend stimulates blood circulation and has anti-inflammatory, anti-oxidant, anti-bacterial, antimicrobial, antiviral, antifungal, analgesic, and hypoallergenic properties. The method of making the formulation ensures proper emulsification, a long shelf life, as well as a proper balance of the various ingredients to enhance effectiveness.

### CROSS REFERENCE TO RELATED APPLICATION

This application claims the priority date of U.S. Provisional Application No. 62/697,239 filed on Jul. 12, 2019.

### BACKGROUND OF THE INVENTION

### 1. Field of the Invention

The present invention relates to topical hair and skin preparations. More specifically, the invention relates to a topical hair and skin formulation which uses no synthetic ingredients and has a long shelf life.

### 2. Description of the Prior Art

Hair and skin preparations have been used by people for millenia for both cosmetic and medicinal purposes. Initially all such preparations were all natural but as technology improved and the need for more effective blends with a longer shelf life arose, synthetics have been added to most if not all such preparations. The reason for this is twofold, the need to effectively emulsify the preparation due to the use of oils/liquids of differing viscosity and other properties; and the need to ensure a long shelf life.

Typically synthetic oils/liquids have been used as both emulsifier and preservative. The problem with the use of such synthetic additives is manifold. First, there is almost always some mild toxicity so that repeated use over a long period can cause problems. Secondly, there is the possibility of allergic reactions for some users. Finally, some people just prefer all natural products.

Therefore, it is an objective of the invention to provide a topical hair and skin formulation which has primarily medicinal and healing properties, but may also be used to moisturize skin and dress or style hair of all types. The formulation is a blend of oils each having various individual properties. The invented oil blend keeps connective tissues strong and supple while protecting against the breakdown of the skin's cell structure and the hair's follicle and inner cuticle with essential vitamins, minerals, amino and fatty acids implementing healing and regeneration. The invented oil blend stimulates blood circulation and has anti-inflammatory, anti-oxidant, anti-bacterial, antimicrobial, antiviral, antifungal, analgesic, and hypoallergenic properties. The method of making the formulation ensures proper emulsification, a long shelf life, as well as a proper balance of the various ingredients to enhance effectiveness.

### SUMMARY OF THE INVENTION

It is a major object of the invention to provide a hair and skin formulation which has medicinal and cosmetic properties.

It is another object of the invention to provide a hair and skin formulation which is a blend of natural oils.

It is another object of the invention to provide a hair and skin formulation which is anti-inflammatory.

It is another object of the invention to provide a hair and skin formulation which is antimicrobial.

It is another object of the invention to provide a hair and skin formulation which is anti-oxidant.

It is another object of the invention to provide a hair and skin formulation which is anti-fungal.

It is another object of the invention to provide a hair and skin formulation which is hypo-allergenic and has analgesic properties.

### DETAILED DESCRIPTION OF THE PREFERRED EMBODIMENTS

The hair and skin formulation of the invention is an all natural blend of active ingredients for effecting the healing, medicinal and cosmetic objectives of the invention, as well as at least one emulsifying and preserving agent. The emulsifier/antioxidant (preserving agent) is also a natural plant based ingredient.

The various oils/substances used in the preparation are known for their respective qualities, e.g., anti-bacterial, anti-inflammatory, etc., some of which are “overlapping” but it is the method of making the inventive formulation, as well as the precise ratios to be discussed below that allow the formulation to be more effective than the ingredients themselves. The inventive formulation is a blend of coconut (Cocos nucifera) oil, avocado (Persea americana) oil, jojoba (Simmondsia chinensis) oil, tamanu (Calophyillum tacamahaca) oil, mixed tocopherol 90% oil, and Glyceryl monostearate.

Coconut oil has anti-inflammatory, antioxidant, anti-bacterial and hydrating properties and is a primary component. Coconut oil contains antioxidant Vitamin E, known to protect skin cells from damage, as well as 3 fatty acids: capric acid, lauric acid and caprylic acid. Each of these acids possess both disinfectant and antimicrobial properties, helping to protect against as well as heal microbial infections such as candida, a common cause of fungal infection. Virgin coconut oil is 44.6% lauric acid, 16.8% myristic acid, and 8.2% palmitic acid, and it's composed mostly of medium-chain-fatty-acids (MCFA). MCFAs are widely regarded as extremely useful for providing sustainable energy due to their molecular structure. Coconut oil contains proteins that contribute to cellular health and tissue repair, along with a wide range of other essential activities within the body.

Avocado oil has anti-inflammatory, antioxidant, and anti-bacterial. Avocado oil is rich in vitamins A, B1, B2, C, D, E, K, and PP as well as potassium, lecithin, and chlorophyll. Avocado oil contains amino acids, sterols, pantothenic acid, lecithin and protein. It also has a high sterolin content which are anti-inflammatory and immune modulating. Much of the essential fatty acids found in avocado oil are monounsaturated oleic acid, the same as omega-9 EFA. Oleic acid is known for reducing inflammation. Avocado oil penetrates deep into the epidermis layers and helps dehydrated skin by providing moisture. Avocado oil can help heal sun damage and age spots, as well as blemishes and eczema.

Jojoba oil has anti-inflammatory, anti-oxidant, anti-bacterial, antimicrobial, antiviral, antifungal, analgesic and hypoallergenic properties. Jojoba Oil has vitamin E, B-complex, copper, zinc, selenium, iodine, and chromium which are essential for healthy hair and skin. Jojoba oil includes palmitic, palmitoleic, stearic, oleic, linoleic, linolenic, and arachidic, eicosenoic, behenic, and erucic and lignoceric fatty acids as well as possessing sulfur, copper, cobalt and has traces of tin. Jojoba oil contains myristic acid which has anti-inflammatory effects. In addition, the chemical composition of jojoba closely resembles that of the skin's natural sebum, so it is easily absorbed and rarely causes allergic reactions, even in the most sensitive individuals. Jojoba oil helps heal acne, pimples and warts, mouth sores, cuts, athlete's foot. Jojoba oil helps to remove build up around hair follicles preventing dandruff, hair loss and dry scalp.

Tamanu oil has anti-inflammatory, anti-oxidant, and anti-bacterial properties plus possesses a unique ability to stimulate the growth of healthy new skin tissue. Tamanu oil is one of the most effective agents in promoting the formation of new tissue known as cicatrization, which means skin regeneration, that accelerates wound healing that inhibit skin tumor production. Tamanu oil contains 3 essential classes of lipids: neutral, glycolipids, and phospholipids. Additionally, the Tamanu oil contains 3 unique and novel compounds that are calophyllic acid, calophyllolide (anti-inflammatory), and lactone (antibiotic). Tamanu oil has active ingredients of friedelin, Inophyllums B and P, Terpenic essences, Benzoic and 0xi-benzoic Acids, Phospho-amino-lipids, Glycerides, Saturated fatty acids, and 4-Phenylcoumarins. Tamanu also has coumarins which is another anti-inflammatory agent. Additionally, tamanu oil was shown to exhibit significant UV absorption capabilities and had a sun protection factor SPF) of 18 to 22.

Natural mixed tocopherol 90% oil is a clear, yellow to brownish-red viscous oil derived from edible vegetable oils (e.g. soybean, canola, sunflower, corn). Natural mixed tocopherol 90% oil is a clear brownish-red viscous oil with a mild taste and odor. Natural mixed tocopherol 90% oil differs from other tocopherol oils by having a maximum soy bean oil content of 10% and a minimum nature mixed tocopherols content of 90%. Natural mixed tocopherol 90% oil has a full spectrum of natural vitamin E, consisting of all 4 major natural Vitamin E homologues: d-alpha-, d-beta-, d-delta-, and d-gamma-tocopherol (naturally occurring biologically active forms of Vitamin E). Note: The gamma tocopherol within quenches peroxynitrite, the free radical that plays a major role in the development of age-related decline. The natural mixed tocopherol oil is an anti-oxidant and is used to control the oxidation of the inventive formulation. It should be noted that the natural mixed tocopherol is also used to control the penetration and stimulation of the oils into the skin and hair.

Distilled Monoglyceride (also known as Glyceryl monostearate) is an emulsifier which is non-hydrogenated and has properties that improves oil blend quality while extending shelf life.

The topical skin and hair formulation of the present invention is an oil blend incorporating, in the percentages described fully below, the various oils discussed above. The formulation may be used as a hair dressing for purely stylistic purposes but is intended nonetheless to provide the healing and prophylactic benefits as described above. The formulation may also be used, for example, as a basic skin moisturizer, as sun block, on small cuts and abrasions, or for any circumstances where the beneficial properties of the oil blend would be effective. The formulation incorporates coconut oil which is solid below about 70 degrees Fahrenheit and a solid or semi-solid mixture is undesirable for consumer use. The formulation was varied to make the oil blend mixture have a “silky” consistency on the skin while ensuring the blend was liquid at room temperature. It was determined that about 49 vol. % coconut oil, 44 vol. % avocado oil, 5 vol. % Jojoba oil, and 1 vol. % tamanu oil was the preferred blend and stayed liquid at room temperature. A preferred range of coconut oil is from about 40 to 60 vol. %. The preferred volume is about 50 vol. %. A preferred range of avocado oil is from about 40 to 60 vol. %. The preferred volume is about 42 vol. %. A preferred range of jojoba oil is from about 2 to 20 vol. %. The preferred volume is about 5 vol. %. A preferred range of tamanu oil is from about 0.5 to 10 vol. %. The preferred volume is about 1 vol. %.

As stated above, natural mixed tocopherol 90% oil is used as an antioxidant. The preferred range of mixed tocopherol 90% oil in the mixture is from about 0.01 to 5% by volume. The preferred volume for hair is about 0.2%, and for skin is about 0.02%.

As previously stated, distilled monoglyceride is used as an emulsifier. A preferred range of distilled monoglyceride is from about 0.01 to 5 vol. %. The preferred volume for hair is about 0.3 vol. % due to the higher antioxidant vol. % and the preferred volume for skin is about 0.03 vol. % due to the lower antioxidant vol. % used within the oil blends.

To prepare the formulation, the coconut oil, avocado oil, Jojoba oil, tamanu oil, natural mixed 90% tocopherol, and distilled monoglyceride are mixed, in the above noted percentages, at between 86 degrees Fahrenheit (30 degrees Celsius) and 140 degrees Fahrenheit (60 degrees Celsius) to bind together the oils, antioxidant, and emulsifier. The preferred blending temperature is approximately 100 degrees Fahrenheit (37.778 degrees Celsius). The oil blend is mixed together with a mixing device until the emulsifier granules are completely dissolved within the oil mixture. Blending time varies depending upon the amount of emulsifier and oils being blended as would be apparent to one of skill on the art.

It is to be understood that the present invention is not limited to the sole embodiment described above, but encompasses any and all embodiments within the scope of the following claims:

I claim:

1. A topical formulation for application to skin or hair comprising:
coconut oil, jojoba oil, avocado oil, tamanu oil, natural mixed 90% tocopherol, and distilled monoglyceride.

2. The formulation of claim 1 wherein said coconut oil is in amount from about 40% to 60% by volume, said avocado oil is in amount from about 40% to 60% by volume, said jojoba oil is in amount from about 2% to 20% by volume, said tamanu oil is in amount from about 5% to 10% by volume, said natural mixed 90% tocopherol oil is in amount from 0.01% to 5% by volume, and said distilled monoglyceride is in amount from 0.01% to 5% by volume.

3. A method of making a topical formulation for application to skin or hair comprising the steps of:
heating a mixture of coconut oil, avocado oil, jojoba oil, tamanu oil, natural mixed 90% tocopherol, and granulated distilled monoglyceride; and,
stirring said mixture at between 86 and 140 degrees Fahrenheit until said granulated distilled monoglyceride is liquid.
